(19) United States
(12) Patent Application Publication
(10) Pub. No.: US 2014/0094676 A1
(43) Pub. Date: Apr. 3, 2014

(54) FLEXIBLE, LIGHTWEIGHT PHYSIOLOGICAL MONITOR

(71) Applicant: CARDIAC INSIGHT, INC., Bellevue, WA (US)

(72) Inventors: Matthew Joseph GANI, Seattle, WA (US); Jay Michael MIAZGA, Seattle, WA (US)

(73) Assignee: CARDIAC INSIGHT, INC., Bellevue, WA (US)

(21) Appl. No.: 14/040,254

(22) Filed: Sep. 27, 2013

Related U.S. Application Data
(60) Provisional application No. 61/707,667, filed on Sep. 28, 2012.

(51) Int. Cl. A61B 5/00

(52) U.S. Cl. CPC A61B 5/683
USPC 600/386

(57) ABSTRACT

Wearable (ambulatory) monitors of the present invention have a segmented design, with at least two and preferably three (or more) mechanically independent, spaced apart sensors (e.g., electrodes or other types of physiological sensors) located in discrete islands, or housing structures that are flexibly connected to one another. Each of the sensors is in operable communication with one or more electronics module(s), also located in one or more of the islands. In some embodiments, an electronics module may be centrally located with respect to two or more peripherally located sensors.

### REFERENCE TO RELATED APPLICATION

This application claims priority to U.S. Provisional Patent Application No. 61/707,667 filed Sep. 28, 2012. The priority application is incorporated by reference in this application in its entirety.

### FIELD AND BACKGROUND

Remote patient monitoring techniques are generally known in which electrodes are mounted on the patient to monitor the patient's vital signs and the detected patient data is transmitted to a remote control module for monitoring the patient's condition. U.S. Pat. No. 7,630,756, for example, discloses a system having electrodes connected to an ambulatory, portable monitoring device that may be carried or worn by the patient, as well as an integrated portable atrial fibrillation monitoring and detection device including a processing component and at least two integrated electrodes built into the body of an integrated monitoring device that can be adhered to the patient.

U.S. Patent Pub'n US 2003/0083559 A1 shows a low profile peripheral monitor patch having a flexible substrate for attachment to a patient and including a high capacity memory for storing and later retrieving sensed and compressed physiological data sensed by electrodes. U.S. Pat. No. 6,580,942 discloses a heart activity detection device having flexible strips, or wings extending from a housing enclosing sensors and circuitry. U.S. Pat. No. 6,416,471 discloses a cordless, disposable sensor band for detecting vital sign data and transmitting it to a remote monitoring station.

Although such devices are known, delivering robust monitoring capability in a device that can be worn by a subject and reliably collect data for an extended period of time has proven difficult. Devices of the present invention are directed to this challenge.

### SUMMARY

Physiological monitors of the present disclosure may be used for monitoring one or more physiological parameters detectable from a surface, such as a skin surface, of a human subject. Such physiological monitors are preferably flexible, lightweight, generally gas and water impermeable, easily adherable to and removable from a patient's skin, integrated (e.g., cordless), and comfortably wearable by a subject for hours, days, weeks, or longer periods without experiencing any degradation in the ability to collect physiological data. In one specific embodiment, monitors of the present invention comprise integrated, portable, wearable atrial fibrillation monitoring devices. In other embodiments, different types of sensors may be implemented and, in some embodiments, multi-parameter monitors may be provided. A monitor weighing about one-fourth of an ounce having a flexible circuit board and multiple ECG electrodes arranged in a segmented, three island configuration has been constructed.

### BRIEF DESCRIPTION OF THE DRAWINGS

FIG. 1 shows a perspective view of a monitor embodiment of the present invention comprising a housing component for mounting an electronics component (shown on the right) and containing a connector circuit to a three electrode patch (shown on the left).

FIGS. 2A-2C illustrate one embodiment of a three island monitor having 3 sensors (e.g., contacts or electrodes), each sensor being associated with an independent, discrete island (housing).

FIGS. 3A-3C illustrate another embodiment of a three island monitor design similar to that shown in FIGS. 2A-2C, but additionally providing a tear-away tab on the underside of the lower, patient contact substrate.

FIGS. 4A-4C illustrate another embodiment (top view, side view and bottom view, respectively) of a three island monitor that is partially disposable and partially re-usable.

FIG. 5 shows an exemplary layout of an additional embodiment of monitors of the present invention.

### DETAILED DESCRIPTION

“Wearable” (ambulatory) monitors of the present invention preferably have a segmented design, with at least two and preferably three (or more) mechanically independent, spaced apart sensors (e.g., electrodes or other types of physiological sensors) located in discrete islands, or housing structures, flexibly connected to one another. Each of the sensors is in operable communication with one or more electronics module(s), also located in one or more of the islands. In one embodiment, an electronics module may be centrally located with respect to two or more peripherally located sensors. In some embodiments, one or more flexible circuit boards may be used. In some embodiments, rigid or substantially rigid electronics and sensor components may be used and located in discrete islands or housing structures, operably connected to one another via flexible wiring.

Each of the housing structures generally encloses an internal volume that may contain one or more sensor(s) and, optionally, other device components. Each of the housing structures provides an internal volume substantially independent of and at least partially sealed with respect to the other housing structures. In some embodiments, one or more of the internal volumes provided in housing structures may be substantially sealed, and in some embodiments, one or more of the islands, or housing structures may enclose multiple substantially discrete or independent internal volumes. In one embodiment, housing structures are formed by joining an upper housing component with a lower substrate that, in operation, contacts and adheres to a monitoring surface, such as a subject's skin. The lower substrate may be provided with one or more ports providing access for the sensor(s) to (directly or indirectly) contact the subject's body surface when contact is required. In embodiments that incorporate sensors that do not require contact (direct or indirect), no such ports are required.

The sensors (e.g., electrodes) may communicate with one or more electronics module(s) wirelessly or via wires. The electronics module may comprise internal circuitry that includes programmable devices, such as a microcontroller and/or a microprocessor, onboard software or firmware executed by the circuitry, memory, signal conditioning circuitry, timers, alarms, and the like. Switches, indicators, control mechanisms, and the like may be provided. The electronics module or other components of the wearable monitor may have the ability to communicate with an externally located computer, data processor, control system, or the like via wire(s) or wirelessly.

At least the lower substrate of the “wearable” monitor is flexible and generally lightweight, as well as preferably being substantially liquid and gas impermeable. In one embodiment, shown in more detail below, the lower substrate of the monitor is fabricated from a flexible, water and gas-impermeable foam material. The external surface of the lower housing substrate may be provided with or comprise an adhesive material providing consistent, reliable adherence of the external surface of the lower substrate to a surface being monitored, such as a subject's body surface. In some embodiments the adhesive composition may substantially cover the external surface of the lower housing substrate, while in other embodiments the adhesive composition may cover only a portion of the external surface of the lower housing substrate. In one embodiment, for example, the adhesive composition may be present on the external surface of the lower housing substrate at locations corresponding to the island, or housing structures, while the external surfaces of the connecting portions, or bridges are not adhesive. The external surface of the lower substrate may be provided with any type of surface profile (e.g., smooth, rough, etc.) that, in combination with an adhesive composition, improves the reliability and durability of the bonding of the lower contact substrate to the subject's body surface. The lower substrate may comprise a single flexible material (e.g., foam) layer with an adhesive applied to its external, contact surface, or it may comprise a multiple layers having different properties bonded to one another and forming a substantially unitary lower substrate.

The top structure of the monitor is generally provided as one or more three-dimensional components that, in combination with the lower substrate, provide multiple independent internal volumes for housing sensors, circuitry, electronics, controllers, memory components, and the like. The top structure may be provided as one or more flexible structure(s) having a 3D configuration, with raised portions providing internal volumes and accommodating internal components. The internal volumes, or islands formed by the combination of the top structure and the lower substrate may have different volumes, configurations, and the like. In one embodiment, for example, a microprocessor island may have a larger internal volume than peripheral sensor/electrode islands. Several specific monitoring device embodiments are illustrated in the attached figures and described below. These specific embodiments are shown and described for illustrative purposes only and are not intended to limit the scope of the inventions in any way.

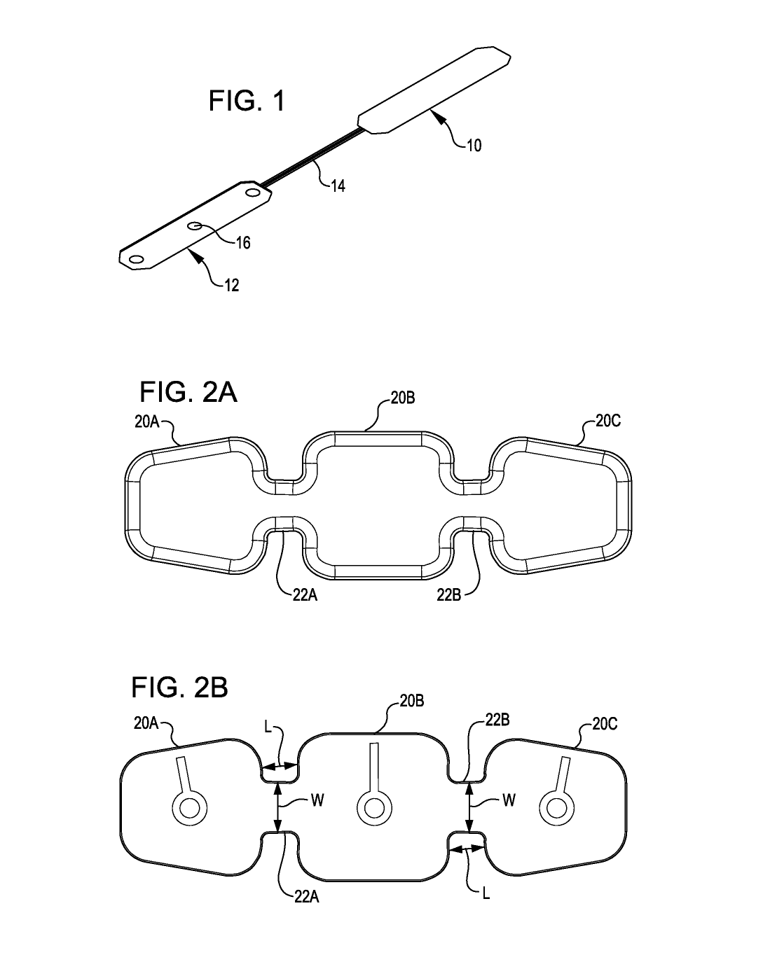

FIG. 1 shows a perspective view of a monitor embodiment of the present invention comprising a housing component 10 for mounting an electronics component (shown on the right) and containing a connector circuit to a three electrode patch 12 (shown on the left). The electronics-containing component and the electrode patch are connected via one or more cable(s) 14 and interface surfaces of each may have an adhesive material. This arrangement desirably and substantially mechanically de-couples the electronics from the electrodes. Three dimensional structures, baffles, or the like may be provided on internal surfaces of either component that, when the electronics-containing component and electrode patch are connected, provide a plurality of substantially discrete internal volumes. The electrode patch 12 typically includes contacts 16 and may be fabricated from typical ECG electrode materials (e.g. gel, metal, etc.) and captured in flexible, adhesive-backed foam for mounting on and sealing to the housing component containing the electronics components. This device is generally provided as a disposable monitor.

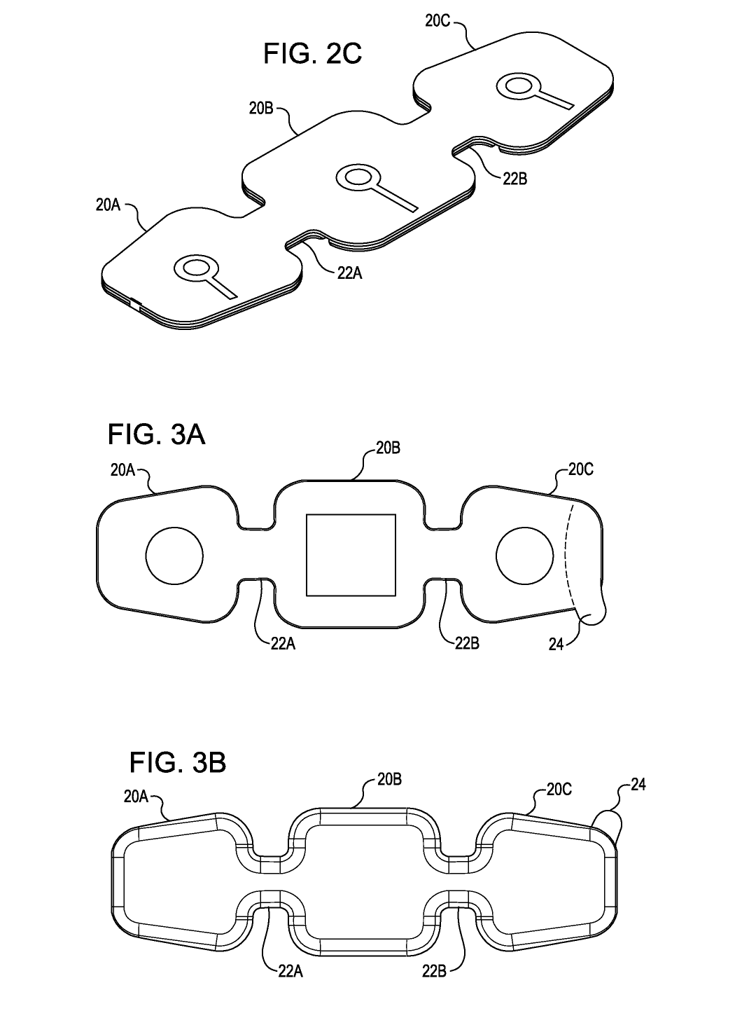

FIGS. 2A-2C illustrate one embodiment of a three island monitor having 3 sensors (e.g., contacts or electrodes), each sensor being associated with an independent, discrete island (housing) illustrated as 20A, 20B and 20C. The islands are separated from and connected to one another by means of narrower flexible portions 22A, 22B. Electronics functions are generally provided in the central island housing 20B. The discrete islands, and the thin, flexible joining portions are formed by a two part housing construction in which a lower, patient contact substrate is generally flat and comprises foam or another flexible, lightweight, non-electrically conductive material. The upper housing component is joined or bonded to the lower substrate along the perimeter of each of the islands, and at the narrower flexible portions. The borders shown along the periphery of the device in FIG. 2A illustrate one exemplary bonding pattern. In another embodiment, the upper and lower housing components may be bonded to one another substantially continuously at the narrower joining portions to provide discrete, substantially water-tight internal island volumes. The upper housing component has a 3D configuration and may be fabricated from flexible, generally lightweight and substantially water and gas impermeable materials, such as flexible foam components.

The narrower flexible portions 22A, 22B provided between islands generally have a width W that is less than about 80%, in some embodiments less than about 60% and in other embodiments less than about 50% the maximum width of a neighboring island. The narrower flexible portions generally have a length L that is less than about 50%, in some embodiments less than about 40% and in other embodiments less than about 30% or 25% of the maximum length of a neighboring island. Sensors for contacting a subject's surface (directly or indirectly) are exposed from the lower contact surface, as shown in FIGS. 2B and 2C. This device is generally provided as a disposable monitor.

In the embodiment illustrated in FIGS. 2A-2C, the central island is generally rectangular and the neighboring, peripheral islands have a generally trapezoidal configuration with a wider dimension nearer the central island and a narrower dimension more distant from the central island. In this embodiment, the central island generally contains the processing functions, although processing functions may additionally or alternatively be provided in other locations.

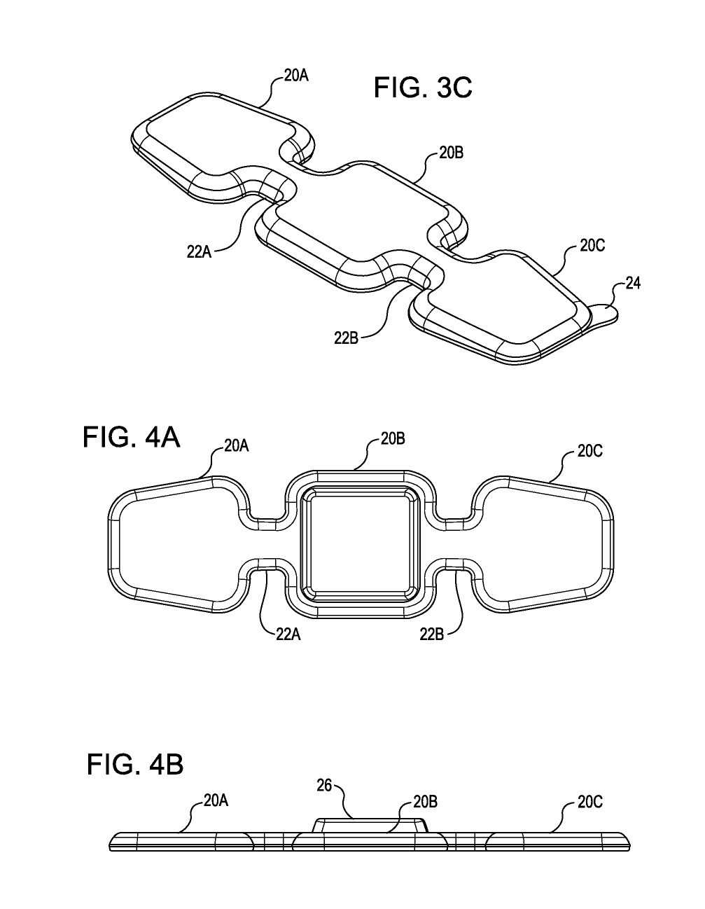

FIGS. 3A-3C illustrate another embodiment of a three island monitor design similar to that shown in FIGS. 2A-2C, but additionally providing a tear-away tab 24 on the underside of the lower, patient contact substrate. The tear-away tab, prior to removal, overlies and protects data contacts while the monitor is adhered to the patient. Following use, the tab is removed, exposing data contacts for retrieval of data from the device. Various types of mating and interfacing systems may be used to transfer data from the electronics module to a monitoring station, device, or the like, and/or to transfer instructions, programming and/or patient information to the electronics module.

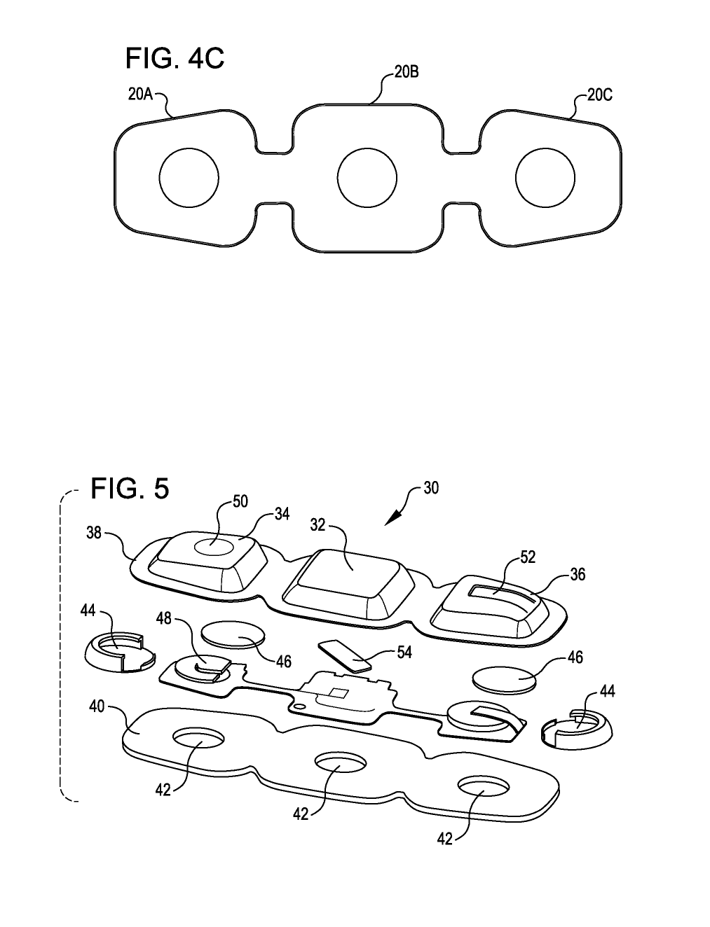

FIGS. 4A-4C illustrate another embodiment (top view, side view and bottom view, respectively) of a three island monitor that is partially disposable and partially re-usable. The lower base component, which contains the patient-contacting (and adhering) substrate and the electrodes, is disposable. The base component may additionally contain non-rechargeable, disposable batteries. The upper, or external, surface of the center island comprises a docking receptacle 26 to which a re-usable electronics module can be attached during use and detached for data downloading and/or re-use. The re-usable electronics module may additionally contain rechargeable batteries, or one or more rechargeable batteries may be provided separately and detachably docked on an external surface of one or more of the device islands. After use, the base component is removed from the subject and disposed of. Once patient data is extracted from the electronics module, it may be used (e.g., docked) in combination with a new base component.

FIG. 5 shows an exemplary layout of additional embodiments of monitors 30 of the present invention. These embodiments use a multiple island design in which a central island 32 contains the processing module and at least two peripheral islands 34, 36 contain a battery and/or accessory components operably linked to the processing module in the central module. In the embodiment shown, the islands are arranged in a generally linear, segmented fashion, with each of the islands having a separate housing with a configuration and volume sufficient to enclose the internal components provided in that housing. The islands (housings) may have different configurations (as shown), different internal volumes, and the like. The segmented islands, or housings, are preferably formed integrally with one another (formed, e.g., by joining external upper and lower foam components 38, 40, respectively) and each of the discrete islands (housings) is preferably joined to at least one other island (housing) via a narrower (or indented) linker section.

In this embodiment, a sensor (e.g., an electrode) is associated with each of the islands and hydrogel wells 42 are provided in ports of the lower substrate 40 in locations corresponding to the sensors. Battery clips 44 may be provided to mechanically hold and maintain contact of the battery(ies) 46 with the associated circuitry. A switch 48 may be provided in one of the internal volumes formed by an island housing for switching the monitor on and off. Additional switches, control features and/or monitors may be provided. The external surface of the island housing may be provided with a surface indication, scored or otherwise marked, as shown at switch location recess 50, to provide an indication of the location of the switch. A data download and/or upload component may be provided in one or more of the internal volumes formed by an island housing that may be exposed by removing all or a part of the housing structure (e.g., tearing away the foam) to expose the data download/upload component(s). The external surface of the island housing may be scored or otherwise marked, as shown at tab location indicator 52, to provide an indication of the location of the data download/upload component. Additional tabs or other access mechanisms may additionally or alternatively be provided for accessing one or more of the internal volumes.

An actuation switch may be provided and made accessible by the subject or a healthcare professional for actuating and/or turning off the device. In one embodiment, an actuation switch may be located within a recess of the battery clip to prevent accidental actuation when the monitor is bumped or the subject lies on it. In one embodiment, a momentary contact switch may be formed by bending a portion of a flexible circuit back on itself, thus putting the two contacts of the switch in opposition, using the inherent spring-like behavior of a bent flex circuit to hold the switch open until it's closed by pressure resulting, for example, from manual contact. Something relatively small in diameter (for instance, the tip of a finger) may be used to actuate the switch inside its recess. Other types of switches, such as electronic microswitches, magnetically actuated switches, and the like may also be used. In some embodiments, a visual indicator such as an LED, or an audible indicator, may be provided for indicating to the subject, or a healthcare provider, that the monitoring device is operational and actuated. In some embodiments, a moisture or water ingress sensor 54 may be provided in one or more of the internal volumes. Indicators may be associated with the moisture sensor(s) to provide alerts indicating moisture intrusion and/or device malfunctions.

It will be appreciated that while specific embodiments have been described and shown, many variations may be implemented without departing from the spirit and scope of this invention. Some embodiments, for example, may employ a two island design, while other embodiments may employ a multiple island design having a symmetrical or asymmetrical arrangement of islands. Sensors may be associated with one or more of the islands, and different types of sensors may be employed to provide a multi-parameter device. While the lower, patient contact/adhering substrate is illustrated as being substantially flat, in alternative embodiments, a patient contact surface of the lower substrate may have a three dimensional configuration in which only portions of the substrate contact a patient's skin surface.

We claim:

1. A physiological monitor comprising a flexible lower substrate configured for adherence to a skin surface, an upper housing providing, in combination with the lower substrate, a plurality of substantially independent, spaced apart internal volumes, at least one sensor provided in at least one internal volume, and at least one electronics module operably connected to the at least one sensor.

2. The physiological monitor of claim 1, wherein the electronics module is provided in an internal volume separate from the at least one sensor.

3. The physiological monitor of claim 1 having a segmented design in which at least three islands are flexibly connected to one another and comprising at least two sensors.

4. The physiological monitor of claim 3, in which a central island houses the at least one electronics module and at least two peripheral islands house sensors.

5. The physiological monitor of claim 3, in which each of the islands is connected to a neighboring island by means of a narrower flexible portion.

6. The physiological monitor of claim 3, comprising an atrial fibrillation monitor.

7. The physiological monitor of claim 1, wherein the upper housing has a three-dimensional configuration with raised portions providing the internal volumes.

8. The physiological monitor of claim 1, wherein at least one of the plurality of substantially independent, spaced apart internal volumes houses a microprocessor and has a larger internal volume than other volumes housing sensors.

9. The physiological monitor of claim 1, wherein the flexible lower substrate is fabricated from a flexible foam material.

10. The physiological monitor of claim 1, wherein the upper housing has a 3D configuration and is fabricated from flexible foam material.

11. The physiological monitor of claim 1, wherein an external surface of the flexible lower substrate is provided with an adhesive material.

12. The physiological monitor of claim 10, wherein the adhesive material covers only a portion of the external surface of the flexible lower substrate.

13. The physiological monitor of claim 1, wherein a central island is generally rectangular and neighboring, peripheral islands have a generally trapezoidal configuration.

14. The physiological monitor of claim 1, wherein a central island houses processing functions.

15. The physiological monitor of claim 1, additionally comprising a tear-away tab overlying data contacts for retrieval of data.

16. The physiological monitor of claim 1, additionally comprising a switch for switching the monitor on and off.

17. The physiological monitor of claim 1, additionally comprising a moisture ingress sensor provided in at least one of the internal volumes.

18. The physiological monitor of claim 1, wherein the at least one sensor comprises at least one ECG electrode.

19. The physiological monitor of claim 1, additionally comprising a docking receptacle for receiving a re-usable electronics module and/or one or more rechargeable batteries.
